(19) United States
(12) Patent Application Publication
(10) Pub. No.: US 2005/0112215 A1
(43) Pub. Date: May 26, 2005

(54) Cramp bark extract and method of extraction

(76) Inventors: Valerie Otto, New York, NY (US); Dinesh Patel, Tuscon, AZ (US); Val Krukonis, Lawrence, MA (US)

Correspondence Address: Peter S. Canelias;Law Offices of Peter S. Canelias, Suite 2148, 420 Lexington Avenue, New York, NY, 10170, US

(21) Appl. No.: 10/961,421

(22) Filed: Oct. 8, 2004

Related U.S. Application Data
(60) Provisional application No. 60/510,017, filed on Oct. 9, 2003.

(51) Int. Cl. A61K 35/78

(52) U.S. Cl. 424/725

(57) ABSTRACT

In cramp bark preparations or tinctures currently available, there are no such preparations that have been standardized to a marker compound. In this type of standardization, one compound serves as a marker to indicate the presence of an identified active ingredient as well as the presence of other constituents that give the herb its therapeutic properties. The invention identifies a compound and a process by which to standardize preparations of cramp bark (Verbena Officinalis) and related species such as black haw, of the Genus Viburnum of the Caprifoliaceae family (herein cramp bark). Verbenalin is an iridoid glycoside, and an alkaloid as well. The extraction process enriches or fortifies the yield of aglycon alkaloids and alkaloid glycosides in the botanical material of the Genus Viburnum family, including cramp bark. Alkaloid glycosides include, without limitation, verbenalin, hastatoside, and verbacoside.

This application claims priority under 35 U.S.C. 120 from provisional application No. 60/510,017, filed Oct. 9, 2003.

### BACKGROUND OF THE INVENTION

Even though approximately 75% of American women of reproductive age experience premenstrual syndrome (PMS), there is no precise medical explanation. PMS is defined as a cluster of physical and emotional symptoms that appear on a regular basis sometime after ovulation and continue until menstrual bleeding begins. At least 150 different symptoms have been associated with PMS. Generally speaking, symptoms are either psychological (such as irritability, mood swings, forgetfulness, and anxiety) or physiological. (such as cramping, bloating, breast tenderness, and headaches).

Studies suggest many different theories. Causes of PMS symptoms have been linked to abnormal hormone levels such as excessive estrogen or deficient progesterone, inappropriate bodily response to hormones, nutritional deficiencies, and various psychological factors.

This invention addresses one of the most common symptoms, menstrual cramping. Menstrual cramps may begin up to seven days before menstruation and continue four days into a woman's period. Menstrual cramps are believed to be caused by an increased secretion of prostaglandins, primarily from the uterus. This can increase inflammation of the tissues as well as the strength of the contraction of smooth muscle. The present invention can also be used for preparation of a remedy for any type of muscle cramping or soreness.

The most frequently used remedy is one of the many nonsteroidal anti-inflammatory drugs (NSAIDs). NSAID's block prostaglandins and are usually the first drugs tried for almost any kind of minor pain. Aspirin is the most common. Others available over the counter are ibuprofen (sold under the brand names Motrin®, Advil®, Nuprin® and Rufen®) and naproxen (sold under the brand name Aleve®). Ibuprofen is one of the most widely used agents for PMS symptoms. Studies have also indicated that naproxen is helpful when started seven days from menstruation and continued for four days into the cycle.

Historically, Viburnum Opulus, also known as Cramp Bark, Guelder rose, or High Cranberry has been used as an antispasmodic; with particular use in relieving muscular and uterine muscle problems. For decades, cramp bark has remained a staple in herbal practice and is specifically indicated in helping to facilitate childbirth, correcting irregular uterine contractions and relieving dysmenorrhoea or severe menstrual cramps.

Several compounds have been reported as being involved with the muscle relaxant activity of cramp bark, they include coumarins, scopoletin, esculetin, viopudal, hydroquinones, tannins (mainly catechins) and vibumin(e) further identified as verbenalin. The most popular theory is scopoletin and other cumarins are responsible for muscle relaxant activity.

In the work by M. Deepak and S. Handa, they have identified a known iridoid glycoside called verbenalin, known for its bitter properties. This is confirmed in work done in 2000 by M. I. Calvo and A. Crespo et al (Acta Horticulture) who point to verbenalin as responsible for the plant's pharmacological activity. Interestingly, viburnine, very close in name, is the compound that is most often named as the active compound in many published monographs of the herb. According to King's American Dispensatory, viburnine is the name that was given to a “secret nostrum” back in 1898 purporting to be obtained from this plant. Other major chemical constituents identified by both the above works are the iridoid hastatoside and the polyphenol verbascoside.

Applicants believe that viburnine (or vibumin) and verbenalin are actively the same compound as explained further herein. Acting as lexicographers, viburnine (or viburnin) and verbenalin are defined as synonymous and will have the same meaning and effect for all purposes of the present invention.

Animal studies show evidence of anti-spasmotic activity of a cramp bark. Methanolic extract of cramp bark was confirmed in the isolated uterus of rats, which showed a 50% reduction in the number of contractions. (J. Calle, M.Toscano et all, Journal of Ethnopharmacology 1999). In 2000, a study showed that a chloroform extract exhibited an anti-inflammatory activity of 56.0% when compared with the standard drug ibuprofen. (M. Deepak and S. Handa, Phytotherapy Research, 2000).

### SUMMARY OF THE INVENTION

In cramp bark preparations or tinctures currently available, there are no such preparations that have been standardized to a marker compound. In this type of standardization, one compound serves as a marker to indicate the presence of an identified active ingredient as well as the presence of other constituents that give the herb its therapeutic properties. The invention identifies a compound and a process by which to standardize preparations of cramp bark (Verbena officinalis) and related species such as black haw, of the Genus Verburnum (herein cramp bark).

By standardizing cramp bark, there is a direct benefit to the end user. The consumer has a more trustworthy product, an extract that is consistent from batch to batch and capsule to capsule. At the present time, one extract, depending on growing conditions and climate, can have a different efficacy than others made by the same manufacturing process.

### BRIEF DESCRIPTION OF THE DRAWINGS

FIG. 1 is a schematic diagram of the fractionation equipment used in the invention;

### DETAILED DESCRIPTION

The cramp bark plant material of the present invention is wild harvested from a pristine environment is Minnesota. The harvest for the materials took place in the early fall of 2002. The harvested cramp bark is then dried in the shade and ground right before extraction.

It is possible that the invention may be performed using any one of several types of extraction methods. These include Supercritical and Sub-critical Fluid extraction that use solvents and pressure to create a liquid state. Solvents may include CO₂, hydro-flourethene, butane, propane and others that create a liquid under pressure. The invention may also be accomplished using a more traditional extraction process—these methods include alcohol, petrol, methanol, and chloroform among others. The heart of the invention is the isolation of verbenalin or alkaloids as the marker compound and preferably standardizing each batch of extract to contain between 6% and 8% .

The invention relates to cramp bark, which is Verbena Officinalis, and to related species such as black haw (Viburnum Purnifolium) and others that are often substituted, all of which are of the Genus Viburnum, belonging to the family Caprifoliaceae. Verbenalin is an iridoid glycoside, and an alkaloid as well. The extraction process enriches or fortifies the yield of aglycon alkaloids and alkaloid glycosides in the botanical material of the Genus Viburnum family, including cramp bark. Alkaloid glycosides include, without limitation, verbenalin, hastatoside, and verbacoside.

In this case, the cramp bark is extracted using a Supercritical Fluid Extraction method (SFE) by the firm Phasex. SFE uses high pressure gases as solvents and is increasingly being employed industrially for extracting oils, resins and other components, from botanical substrates. SFE makes use of CO₂ gas under high pressure. When CO₂ is at conditions of temperature and pressure above 31° C. and 73 atmospheres (atm), respectively, it is in its supercritical state where it exhibits a pressure-dependent dissolving power for a wide variety of natural products, pharmaceuticals, and polymers. At high pressure the gas dissolves and extracts soluble components (i.e., oils, actives, etc.) from the botanical substrate, and when the pressure is lowered (in another part of the process plant) the dissolved components precipitate and are collected. The product is a completely pure extract, containing no liquid organic solvents. One caveat is that not all botanicals respond well to this type of extraction method, but ones that do are easily identifiable by analyzing the compounds present in the virgin material and comparing them to the compounds known to dissolve in CO₂.

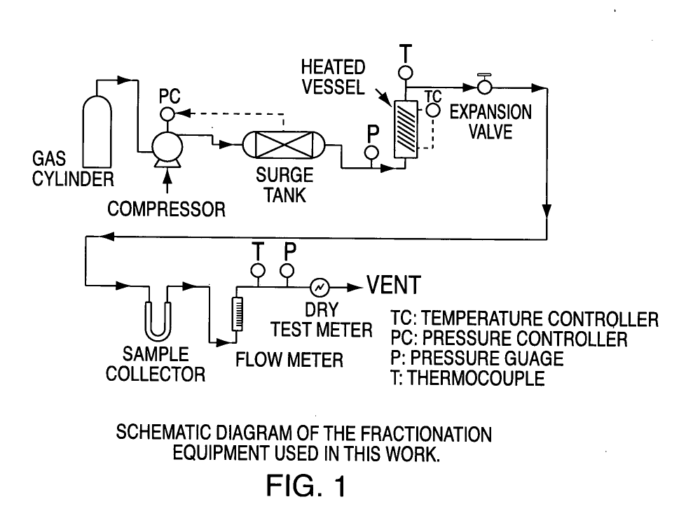

The extraction of actives from cramp bark was carried out in an SFE system shown schematically in FIG. 1 to carry out a supercritical fluid extraction. The cramp bark is charged to an extraction vessel, and CO₂, at selected pressure and temperature conditions is passed through the vessel for a period of time. It is not actually time, but total mass of gas, that is the important variable since the flow rate is adjustable. Conceptually, it is easier to relate to time.

The high pressure stream of gas plus material that is extracted from the cramp bark is passed in series through a pressure reduction valve into a collector where the extractables precipitate. The atmospheric gas exits the flask and flows through a dry test meter for integration of total volume. After flowing a desired amount of gas through the extraction vessel, the flask is removed, the conditions changed and the procedure repeated. Increasing pressure sequentially during a test often allows a selective concentration of different materials, the procedure is referred to as Pressure Profiling. Two pressure levels, one at 2500 pounds per square inch (psi) and a higher one, 5000 psi were tested in some initial small scale (10 g) experiments; it was found (by analyzing the extracts) that 5000 psi gave a higher yield. In one larger scale test 450 g of dried cramp bark were extracted with supercritical CO₂ at conditions of 5000 psi and 60° C. An extract of 72.5 g was obtained. About 50 g was condensed moisture (which also dissolves in CO₂).

Identification and quantification of the verbenalin content of the Phasex cramp bark extract and dosage calculation based on a well known, reputable cramp bark tincture from a health food store is described as follows. Whole Cramp Bark and the Phasex extract (100 mg) were dissolved into tetrahydrofuran:methanol (1:1) 100 ml subjected to soncation for 5 minutes at 30° C. Aliquots were removed, filtered through a PVDF membrane (0.45 μm) and placed in vials for HPLC analysis. The chromatographic separation was achieved using a Cosmosil C18-AR column (150×4.6 mm, 5 μm, 120 Angstrom) and a gradient elution with a mixture of two solvents: solvent A, water/glacial acetic acid/tetrabutylammonium hydrogensulphate (TBAHS) and solvent B, methanol/glacial acetic acid/TBAHS. The effluent was monitored using a Diode Array detector scanning from 200 to 460 nm. Analysis was also conducted by GC/MS with the following conditions: Econowax SP-32 (30 m 0.45 μm id) column initial temperature 70° C., holding 2 mins., ramping rate was 10° C./min linear, to a final temperature 280° C., holding 5 minutes. The samples were additionally scanned by Fisions GC/Voyager MD800 MS with a quadrupole mass filter in an electron impact mode (70 eV) for the volatile components present in the Phasex extract.

[image]

Principle components identified through the chromatographic procedure were then subjected to isolation on a Vydac C-18 preparative column (250×460 mm 50 μm 120 Angstrom) with an identical solvent system at a flow rate of 40 ml/min. Desired peaks were collected from the effluent stream, solvent removed in vacuo and lyophilized to dryness. Samples of the prepared fractions were analyzed by LC/MS after thoroughly dissolving the sample in methanol, filtered through a 0.45 micron PVDF membrane and placed into suitable vials for HPLC-MS analysis.

[image]

The chromatography was performed on a Waters 2695 separation module using a Wako Wakosil-II 5C18 HG column (5 mum, 15 cm*4.6 mm i.d.) at 45° C. with gradient elution of H₂O:methanol (1 ml/min) from 22:3 to 77:23 in 19 min, then to 18:7 at 24 min and 27:23 at 39 min. The chromatographic eluent was passed into a Vestec particle-beam interface for solvent removal and particle atomization and then via Teflon transfer line into the mass spectrometer using a helium carrier gas. Detection was performed on a Finnigan TSQ7000 triple-quadrapole mass-spectrometer in positive ion mode with full scan centroid data collection (50-1000 m/z). MS-MS experiments using an argon collision gas were used to verify the identity of the glycoside alkaloids.

[image]

The principle alkaloid present in the Phasex extract was determined to be verbenalin, based upon an extensive literature review. See Suomi J; Siren H; Jussila M; Wiedmer S K; Riekkola M L: Determination of iridoid glycosides in larvae and adults of butterfly Melitaea cinxia by partial filling micellar electrokinetic capillary chromatography-electrospray ionisation mass spectrometry: ANALYTICAL AND BIOANALYTICAL CHEMISTRY 2003, Vol 376, Iss 6, pp 884-889: 6: July: 700CU:; Suomi J; Wiedmer S K; Jussila M; Riekkola M L: Determination of iridoid glycosides by micellar electrokinetic capillary chromatography-mass spectrometry with use of the partial filling technique: ELECTROPHORESIS 2001, Vol 22, Iss 12, pp 2580-2587: 8: August: 464JQ:; Deepak M; Handa S S: Quantitative determination of the major constituents of Verbena officinalis using high performance thin layer chromatography and high pressure liquid chromatography: PHYTOCHEMICAL ANALYSIS 2000, Vol 11, Iss 6, pp 351-355: 5: November-December: 387QB:; Deepak M; Handa SS: Antiinflammatory activity and chemical composition of extracts of Verbena officinalis: PHYTOTHERAPY RESEARCH 2000, Vol 14, Iss 6, pp 463-465: 3: September: 355XD:; Calvo M I; Vilalta N; San Julian A; Fernandez M: Anti-inflammatory activity of leaf extract of Verbena officinalis L.: PHYTOMEDICINE 1998, Vol 5, Iss 6, pp 465-467: 3: December: 172PE:; Rajnikant; Lal M; Gupta V K; Singh A; Suri O P: Crystal structure of 1 alpha-(beta-D-glucopyranosyloxy)-1,4a alpha,5,6,7,7a alpha-hexahydro-7 alpha-methyl-5-oxocyclopenta[c]pyran-4-carboxylic acid methyl ester (Verbenalin): CRYSTALLOGRAPHY REPORTS 1998, Vol 43, Iss 3, pp 448-453: 6: May-June: 118FM:; Singh B; Saxena A; Chandan B K; Anand K K; Suri O P; Suri K A; Satti N K: Hepatoprotective activity of verbenalin on experimental liver damage in rodents: FITOTERAPIA 1998, Vol 69, Iss 2, pp 135-140: 6: ZV340:; Calvo M I; San Julian A; Fernandez M: Identification of the major compounds in extracts of Verbena officinalis L. (Verbenaceae) by HPLC with post-column derivatization: CHROMATOGRAPHIA 1997, Vol 46, Iss 5-6, pp 241-244: 4: September: XW651:; Recio Md; Giner Rm; Manez S; Rios J I: Structural Considerations On The Iridoids As Antiinflammatory Agents: Planta Medica 1994, Vol 60, Iss 3, Pp 232-234: 3: June: Nt369:; Semenova Op; Timerbaev Ar; Bonn Gk: Application Of High-Performance Liquid-Chromatography To The Determination Of Bitter Principles Of Pharmaceutical Relevance: Journal Of Chromatography A 1994, Vol 667, Iss 1-2, Pp 327-333: 7: April 29: N1080] the structural elucidation is consistent with latest accepted structure postulated in the literature.

The amount of verbenalin was determined to be 5.6 mg. of the 72.5 mg. extract or about 7.7% by weight. This invention uses the above Chromatograms and resulting isolation of cramp bark's most active compound to establish a minimal level that insures efficacy. It must be noted that after an exhaustive literature review on this active 5 compound of cramp bark, there appears to be no difference between the compounds viburnine and verbenalin. The essence of the invention is that we have established a marker compound, verbenalin (or alkaloids), by which to standardize the herb within a preparation. The exact percentage of verbenalin can be varied according to the desired strength, although our analysis shows the extract should contain a minimum of 7%, but 10 again is varied depending on particularities of the subject who will ingest the preparation.

[image]

Initial dosage determination was derived from the total alkaloid concentration present in a daily serving of the reputable cramp bark product. The commercially available tincture was evaporated to dryness, weight of dissolved solids determined and equated to a daily serving. Alkaloid content was determined through HPLC for quantification and through LC/MS for identification. Since the reputable cramp bark product was found to contain very little verbenalin it was deemed acceptable to equate the dosage to the sum of all alkaloids that could be measured in this product.

One day's serving of the reputable cramp bark contained 1.2 mg. of alkaloids (1.2 mg/0.077=15.58 mg of the invention's active ingredient per serving). SCF extractions can not be dried due to their high oil content. As a result the extract must be suspended in an excipient such as canola oil or olive oil that will not interact with the extract. The resulting product is to be put into gelatin capsules. Each serving of the inventors' preferred extract will contain the equivalent of 15 mg or another dosage that is considered safe and therapeutic.

While the invention has been described with references to certain preferred embodiments those skilled in the art will recognize that modifications and variations may be made in construction, material and method without departing from the spirit and scope of the present invention, which is intended to be limited only by the scope of the claims appended hereto.

1. A method of standardizing an active ingredient of a therapeutic compound, comprising the steps of:
employing an extraction method on a material containing botanical matter of a genus viburnum to produce an extract;
measuring an amount of verbenalin as a marker compound to determine a concentration of aglycon alkaloids and alkaloid glycosides in the extract; and
comparing the measured amount of verbenalin to a standard.

2. The method of claim 1, where if the measured amount of verbenalin and thus total amount of aglycon alkaloids and alkaloid glycosides is less than the standard, repeating the process steps until a measured amount of verbenalin is substantially equal to the standard.

3. The method of claim 1, wherein the standard is about 6%-10% by weight.

4. The method of claim 1, wherein the extraction method is a supercritical fluid extraction method.

5. The method of claim 4, wherein the supercritical fluid extraction method employs carbon dioxide gas as a solvent.

6. The method of claim 1 wherein the extraction method is a sub-critical fluid extraction method.

7. The method of claim 1, wherein the extraction method utilizes alcohol as a solvent.

8. The method of claim 1, wherein the extraction method utilizes petrol as a solvent.

9. The method of claim 1, wherein the extraction method utilizes methanol as a solvent.

10. The method of claim 1, wherein the extraction method utilizes chloroform as a solvent.

11. The method of claim 1, wherein the material is a botanical substrate.

12. A method of extracting a therapeutic compound, comprising the steps of:
charging botanical matter of a genus viburnum to an extraction vessel;
passing carbon dioxide gas that is substantially at a selected temperature and substantially at a selected pressure through the extraction vessel to create a stream comprised of the carbon dioxide gas and an extracted material;
passing the stream though a pressure reduction valve into a collector where the extracted material is precipitated;
passing the gas stream through a test meter for integration of a total volume; and
measuring an amount of verbenalin contained in the extracted material.

13. The method of claim 12, wherein the selected pressure of the carbon dioxide gas is about 2500 psi.

14. The method of claim 12, wherein the selected pressure of the carbon dioxide gas is about 5000 psi.

15. The method of claim 12, wherein the selected temperature of the carbon dioxide gas is about 60° C.
